(19) United States
(12) Patent Application Publication
(10) Pub. No.: US 2024/0173157 A1
(43) Pub. Date: May 30, 2024

(54) ORTHOPAEDIC DEVICE, ORTHOPAEDIC KIT AND METHOD

(71) Applicant: Dassiet Oy, Espoo (FI)

(72) Inventors: Jimmy TAKKI, Espoo (FI); Michael LINDROOS, Espoo (FI); Kerkko VISURI, Espoo (FI)

(21) Appl. No.: 18/284,696

(22) PCT Filed: Mar. 28, 2022
(86) PCT No.: PCT/FI2022/050197

§ 371 Date: Sep. 28, 2023

Related U.S. Application Data
(60) Provisional application No. 63/167,229, filed on Mar. 29, 2021.

(51) Int. Cl. A61F 5/01

(52) U.S. Cl. CPC A61F 5/0104

(57) ABSTRACT

An orthopaedic device, a kit comprising said device, and a method. The orthopaedic device comprises a support plate, a piece of fabric. The support plate is malleable into an orthopaedically supporting shape, the support plate having a supporting surface to be arranged towards an object to be supported and an opposite distal surface. The supporting surface of the support plate is adhered to a receiving zone of the piece of fabric, such that the distal surface of the support plate constitutes a part of the distal surface of the device. The piece of fabric is larger than the support plate, providing thus an attachment zone for fastening the orthopaedic device.

### BACKGROUND

The invention relates to an orthopaedic device.

The invention further relates to an orthopaedic kit.

The invention still further relates to a method for providing temporary orthopaedic support or protection.

A problem with known orthopaedic devices is that their use requires high level of skill and craftsmanship. In this disclosure, the term “orthopaedic device” covers devices used in orthopaedic surgery for e.g. prevention and treatment of disorders of the bones, joints, ligaments, tendons and muscles, as well as protective equipment, e.g. braces and supports used in e.g. sports for providing compression, support and/or joint stability.

### BRIEF DESCRIPTION

Viewed from a first aspect, there can be provided an orthopaedic device, comprising a support plate, a piece of fabric, wherein the support plate is malleable into an orthopaedically supporting shape, the support plate having a supporting surface to be arranged towards an object to be supported and an opposite distal surface, wherein the supporting surface of the support plate is adhered to a receiving zone of the piece of fabric, such that the distal surface of the support plate constitutes a part of the distal surface of the device, and the piece of fabric is larger than the support plate (1), providing thus an attachment zone for fastening the orthopaedic device (100).

Thereby an orthopaedic device that is quick and easy to use even by not highly skilled personnel may be achieved.

Viewed from a further aspect, there can be provided an orthopaedic kit, comprising a support plate, and a piece of fabric, wherein the support plate is malleable into an orthopaedically supporting shape, the support plate having a supporting surface to be arranged towards an object to be supported, and an opposite distal surface, wherein the size of the piece of fabric is larger than the size of the support plate, wherein the orthopaedic kit further comprises an instruction document including instructions for shaping of the support plate into the orthopaedically supporting shape, fastening the supporting surface of the support plate to piece of fabric, arranging the supporting surface on its supporting position on the object, and winding the piece of fabric around the object. in full)

Thereby an orthopaedic kit that is quick and easy to use even by not highly skilled personnel may be achieved.

Viewed from a further aspect, there can be provided a method for providing temporary orthopaedic support or protection, comprising:
providing a support plate, and a piece of fabric, wherein the support plate is malleable into an orthopaedically supporting shape, the support plate having a supporting surface to be arranged towards an object to be supported, and an opposite distal surface, and wherein the size of the piece of fabric is larger than the size of the support plate,
shaping the support plate into shape of the object to be supported or protected,
fastening the supporting surface of the support plate to the piece of fabric,
arranging the supporting surface on its supporting position on the object so that the piece of fabric is situated between the support plate and the object, and
fastening the support plate on the object by using the piece of fabric.

Thereby a method that is quick and easy for even not highly skilled personnel may be achieved.

The arrangement, the kit and the method are characterised by what is stated in the independent claims. Some other embodiments are characterised by what is stated in the other claims. Inventive embodiments are also disclosed in the specification and drawings of this patent application. The inventive content of the patent application may also be defined in other ways than defined in the following claims. The inventive content may also be formed of several separate inventions, especially if the invention is examined in the light of expressed or implicit sub-tasks or in view of obtained benefits or benefit groups. Some of the definitions contained in the following claims may then be unnecessary in view of the separate inventive ideas. Features of the different embodiments of the invention may, within the scope of the basic inventive idea, be applied to other embodiments in combination or instead thereof.

In one embodiment, the support plate is made of a composition comprising thermoplastic polymer and wood particles in form of granules, plates and/or fibers.

An advantage is that the material is easy to work with and has a relatively light weight while maintaining the necessary orthopaedic support. A further advantage is that the material may provide a high breathability that promotes healing of wounds and reduces irritation of skin under the support plate. A still further advantage is that the material may be reheated and reshaped, and thus wastage of the material may be reduced.

In one embodiment, the piece of fabric is made of material comprising thermoplastics, preferably of 100% polyester.

An advantage is that a light, breathable and non-wetting piece of fabric may be achieved.

In one embodiment, the piece of fabric has a self-attaching surface structure having ability to fasten with itself as such.

An advantage is that the device is simple to use and number of components thereof may be reduced.

In one embodiment, the self-attaching surface structure of the piece of fabric comprises a fibre loop bundle fabric.

An advantage is that an elastic and soft self-attaching surface structure may be achieved.

In one embodiment, the piece of fabric has ability to fasten with itself in a normal temperature, such as in temperature of 20° C.-25° C.

An advantage is that the orthopaedic device is easy to use at normal room temperatures without need for heating the fabric.

In one embodiment, the orthopaedic device comprises an exposing element arranged for exposing a premature removal of the orthopaedic device.

An advantage is that a wrong use of the device taking place either intentionally or unintentionally may be easily observed.

In one embodiment, the orthopaedic device comprises a further material layer, such as a layer of fabric, fastened to the receiving zone of the piece of fabric, either on same side with or opposite side thereof in relation to the support plate.

An advantage is that a padding between the support plate and the object to be supported may be added, and thus chafing of skin under the support plate lessened. In embodiments where the support plate is heated in a raised temperature and the shaped against the object, an additional heat insulation may be created between the heated support plate and tissue of the object.

In one embodiment, the support plate and the piece of fabric of the kit are specifically shaped and sized for supporting an indication selected from the following indications or body part: humerus, wrist, thumb, elbow, knee, ankle, metatarsal fracture, metacarpal fracture, finger, phalanx, forearm, upper arm, neck, spine.

An advantage is that an optimal support of the object may be created quickly, since the operator needs not to cut the support plate and the piece of fabric into the required shape.

### BRIEF DESCRIPTION OF FIGURES

Some embodiments illustrating the present disclosure are described in more detail in the attached drawings, in which

FIG. 1a is a schematic view of an orthopaedic device and method prior to adhering a plate to a piece of fabric,

FIG. 1b is a schematic view of the orthopaedic device and method shown in FIG. 1a the plate adhered to the piece of fabric,

FIG. 2 is a schematic view of an orthopaedic device and method,

FIGS. 3a to 3d are schematic views of some embodiments of the plate,

FIGS. 4a and 4b are schematic views of some embodiments of the piece of fabric, and

FIG. 5 is a schematic view of a kit comprising the orthopaedic device.

In the figures, some embodiments are shown simplified for the sake of clarity. Similar parts are marked with the same reference numbers in the figures.

### DETAILED DESCRIPTION

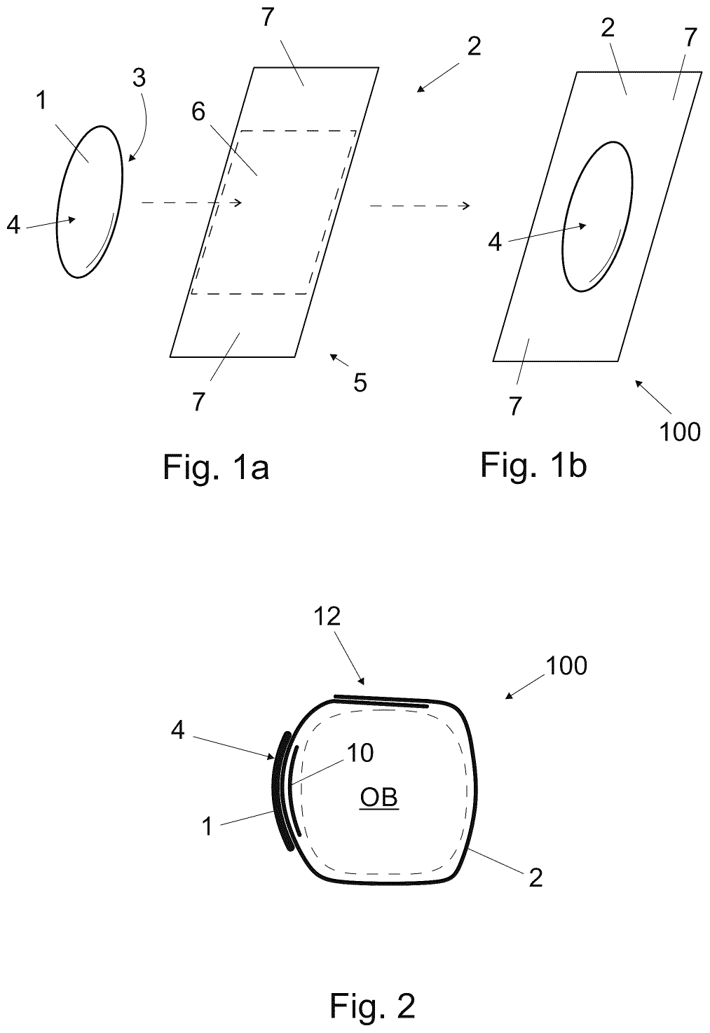

FIG. 1a is a schematic view of an orthopaedic device and method prior to adhering a plate to a piece of fabric, and FIG. 1b is a schematic view of the orthopaedic device and method shown in FIG. 1a the plate adhered to the piece of fabric.

The orthopaedic device 100 comprises a support plate 1 and a piece of fabric 2. The support plate 1 can be shaped in a three-dimensional shape that gives a desired orthopaedical support to an object to be supported. The “object” may be any part of the body of a human being or an animal.

The support plate 1 has two surfaces or sides: a supporting surface 3 to be arranged towards the object to be supported and an opposite distal surface 4.

In one embodiment, the piece of fabric 2 has a self-attaching surface structure 5. This has an ability to fasten with itself as such.

In one embodiment, all the surface area of the piece of fabric contains the self-attaching surface structure 5.

In one embodiment, there are some area (s) or section (s) in the piece of fabric 2 that do not have the self-attaching surface structure 5, or in other words: only part of the surfaces of the piece of fabric 2 comprises the self-attaching surface structure 5.

In one embodiment, the whole piece of fabric 2 is without the self-attaching surface structure 5. Then, the orthopaedic device 100 is secured on the object by e.g. using additional attachment substances or means, such as an adhesive, tape or staples, and/or by tying/knotting the piece of fabric.

The orthopaedic device 100 is constructed by attaching the supporting surface 3 of the support plate to a receiving zone 6 of the piece of fabric.

In one embodiment, the support plate 1 is attached by using an additional attachment substance, such as adhesive, and/or an additional attachment means, such as staples.

In one embodiment, the support plate 1 is attached to the piece of fabric 2 without any additional attachment substance or additional attachment means. For instance, material of the support plate 1 may turn into a tacky state when warmed up and attach to the piece of fabric 2.

The receiving zone 6 is a part of the piece of fabric 2 in which the support plate 1 should be attached for realizing a useful or optimal orthopaedic device 100. The receiving zone 6 may comprise the self-attaching surface structure 5, but it is not necessary.

FIG. 1b is showing an embodiment of a complete orthopaedic device 100. The distal surface 4 of the support plate is a part of a distal surface of the device. The distal surface means the surface that is arranged distally in relation to the supported object. The piece of fabric 2 is larger than the support plate 1. Thus, there is an attachment zone 7, i.e. free surface, on the distal surface that makes it possible the piece of fabric 2 to fasten with itself with said distal surface.

In one embodiment, such as shown in FIG. 1b, the orthopaedic device 100 comprises just one support plate 1 attached in the piece of fabric 2. In another embodiment, there are two or even more discrete support plates 1 attached in one piece of fabric 2.

FIG. 2 is a schematic view of an orthopaedic device and method.

The device 100 is arranged on the object OB so that the support plate 1 is placed on a supporting position on the object OB so that the supporting surface is towards the object OB and the piece of fabric 2 lies between the support plate 1 and the object OB.

In one embodiment, the support plate 1 is shaped to the shape of the object OB by pressing the support plate against the object. The shaping of the support plate can be done prior to attaching the support plate to the piece of fabric 2, simultaneously with attaching the support plate to the piece of fabric, or after attaching the support plate to the piece of fabric.

In one embodiment, the support plate 1 is heated to a higher temperature to ease shaping thereof. For example, support plates manufactured of composition comprising thermoplastic polymer (such as and wood particles in form of granules, plates and/or fibers can be heated to e.g. about 65° C., and then shaped in the required shape. The heating may be carried out e.g. in a water bath, in an oven or in a microwave oven.

The attachment zone 7 has been tied around the object OB such that a suitable compression is created against the object, and the piece of fabric 2 has been fastened with itself on its overlapping sections 12.

In one embodiment, the arrangement 100 comprises at least one further material layer 10, such as a layer of fabric, fastened to the receiving zone 6 of the piece of fabric 2 so that the further material layer 10 lies between the support plate 1 and the object OB. The further material layer 10 may be arranged between the support plate 1 and the piece of fabric 2, or an opposite side of the piece of fabric 2 in relation to the support plate 1. In one embodiment, there are further material layers 10 on both sides of the piece of fabric 2. It is to be noted that the arrangement 100 may comprise further material layer (s) 10 in some other part of the piece of fabric as in the receiving zone 6.

Typically, the support plate 1 has a concave shape towards the object to be supported. One example of this kind of shape is shown in FIG. 2. However, in another embodiments, the support plate 1 may have a convex shape or a planar surface or any combinations of concave, convex and planar shapes towards the object.

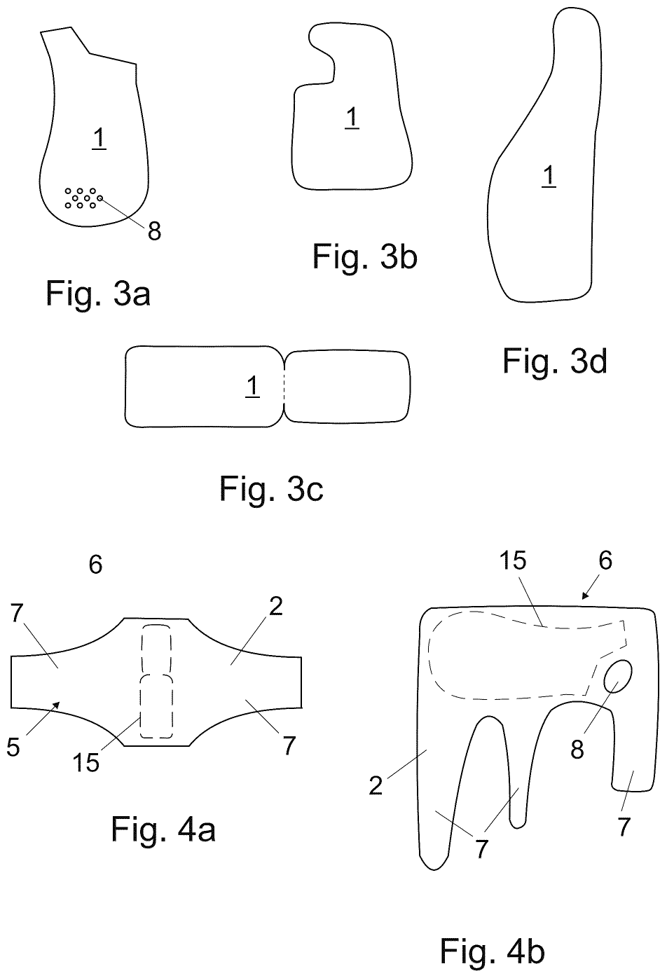

FIGS. 3a to 3d are schematic views of some embodiments of the plate.

In one embodiment, the support plate 1 has a universal shape that is not shaped and sized to a specific object or indication but can be used at least satisfactorily in many applications. Such a shape may be e.g. rectangle or round shape.

In one embodiment, the support plate 1 has a shape that is shaped and sized to a specific object or indication. FIGS. 3a-3d are showing some examples of specifically shaped support plates.

FIG. 3a is showing an embodiment of the support plate intended for supporting a thumb.

In one embodiment, the support plate 1 is solid, i.e. without through holes. In another embodiment, the support plate 1 comprises at least one opening 8, typically plurality of openings. The opening (s) may have various functions, for example promoting breathability of the object covered by the support plate. It is to be noted that the least one opening 8 may be fitted to any support plate 1 described in this disclosure.

In one embodiment, the opening 8 arranged in the support plate 1 may be dimensioned and placed for receiving a body part, such as a finger or toe. The shape of the opening may vary.

FIG. 3b is showing an embodiment of the support plate intended for supporting a wrist from dorsal or palmar direction.

FIG. 3c is showing an embodiment of the support plate intended for supporting an elbow. The support plate 1 can be bent along dashed line to a suitable angle.

FIG. 3d is showing an embodiment of the support plate intended for supporting a hand having a metacarpal fracture.

The orthopaedic device 100 can also be applied to and shaped for other indications, such as: humerus, knee, ankle, wrist, metatarsal, metacarpal, finger, phalanx, forearm, upper arm, neck, spine, etc.

The orthopaedic device 100 can be used for immobilisation of fractures and strains etc. of bones, ligaments, nervous system etc. of human beings and animals.

In one embodiment, the support plate 1 is made of material that comprises metal or polymer material or both. Said metal may be e.g. Aluminium. Said polymer material may be thermoplastic or thermoset material. In one embodiment, said material is a composite material comprising polymer material and reinforcing material. In one embodiment, the polymer material comprises thermoplastic polymer and the reinforcing material comprises wood particles in form of granules, plates and/or fibers. In one embodiment, the thermoplastic polymer is non-biodegradable polymer. In another embodiment, the thermoplastic polymer is biodegradable polymer. In one embodiment, the biodegradable polymer comprises caprolactone homopolymer and the reinforcing material comprises platy wood particles. This composite material is easy to work with and has a light weight. It can also be shaped by using elevated temperature, such as about 65° C., if necessary.

In one embodiment, the thickness of the support plate 1 is in range of 0.8 mm-8 mm, preferably 2 mm-4 mm. The thickness may be equal essentially all over the plate, or there may be variations e.g. for alleviating shaping of the plate.

According to an aspect, the support plate 1 is manufactured by extruding an extrudate that is then cut in suitable pieces. According to another aspect, the support plate 1 is manufactured by casting.

FIGS. 4a and 4b are schematic views of some embodiments of the piece of fabric.

In one embodiment, the piece of fabric 2 has a universal shape that is not shaped and sized to a specific object or indication but can be used at least satisfactorily in many applications. Such a shape may be e.g. rectangle shape.

In one embodiment, the piece of fabric 2 has a shape that is shaped and sized to a specific object or indication. FIGS. 4a, 4b are showing some examples of specifically shaped support plates.

In one embodiment, the piece of fabric 2 is made of material comprising thermoplastics. In one embodiment, the piece of fabric 2 is a textile material made of polyester, such as of 100% polyester. In one embodiment, the piece of fabric 2 comprises polyester yarn of type 50/25 SDY.

In one embodiment, the piece of fabric 2 comprises or is made completely of 3D air mesh-type fabric. An advantage of this type of fabric is high breathability and water permeability. Following this, if the piece of fabric 2 get wet, it will dry quickly, and water does not cause problems to skin under the device.

In one embodiment, the further material layer 10 is made of 3D air mesh-type fabric described above.

The piece of fabric 2 is preferably bi-directionally elastic or stretchy such that the supported object may rotate in an extent that promotes healing of the object.

In one embodiment, the thickness of the piece of fabric 2 is in range of 2 mm-4 mm. The thickness may be equal essentially all over the fabric, or there may be variations in the thickness.

The structure of the piece of fabric 2 is preferably breathable in order to alleviate healing of the object covered by the fabric.

In one embodiment, the self-attaching surface structure 5 of the piece of fabric comprises a fiber loop bundle fabric.

In one embodiment, the self-attaching surface structure 5 of the piece of fabric comprises a hook-and-loop fastener surface (Velcro).

The self-attaching surface structure 5 provides a stepless adjustment of the attachment, and thus e.g. an optimal compression towards the object may be created.

In one embodiment, the piece of fabric 2 can be fasten with itself in a normal temperature, such as in temperature range of 20° C.-25° C.

In one embodiment, the piece of fabric 2 comprises at least one active additive for promoting healing of possible wound (s) relating to the supported object. In one embodiment, the piece of fabric 2 comprises silver or silver alloy particles or fibres.

In one embodiment, such as shown in FIG. 4a, the piece of fabric 2 comprises a guiding marking 15 that guides the operator to place the support plate 1 to a correct position on the fabric.

In one embodiment, the piece of fabric 2 comprises attachment zones 7 on at least two sides of the support plate 1. In another embodiment, the piece of fabric 2 comprises the attachment zone 7 just one side of the support plate 1.

In one embodiment, such as shown in FIG. 4b, the attachment zone 7 is a finger-like projecting part of the piece of fabric 2 that is suited for wrapping around the object to be supported. It is to be noted that the attachment zone 7, regardless of its shape, may be dimensioned so that it is wrappable at least two times around the object to be supported. However, this is not necessary in all embodiments of the device.

The embodiment shown in FIG. 4b comprises three finger-like projecting parts. However, the number of the finger-like projecting parts may vary from one to numbers more than three.

In one embodiment, the piece of fabric 2 comprises at least one opening 8 for e.g. receiving a body part, such as a finger or toe. The shape of the opening may vary.

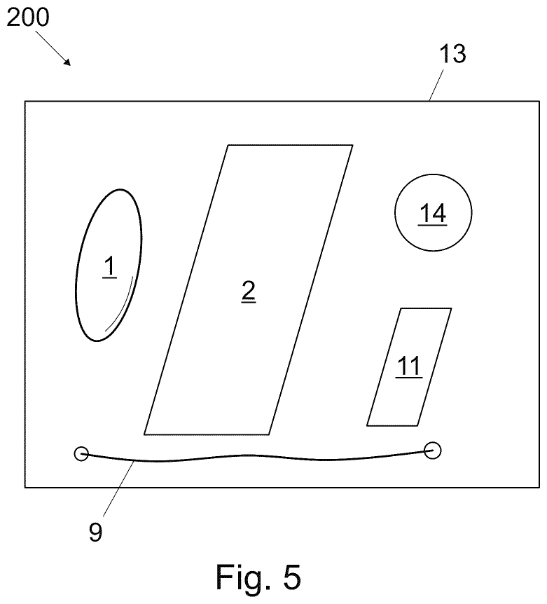

FIG. 5 is a schematic view of a kit comprising the orthopaedic device.

According to an aspect, components of the orthopaedic device 100 according to the current disclosure are arranged in an orthopaedic kit 200. The orthopaedic kit 200 thus comprises the support plate 1 and the piece of fabric 2. The orthopaedic kit 200 comprises also an instruction document 11 including instructions for at least:
a) shaping of the support plate 1 into the orthopaedically supporting shape,
b) fastening the supporting surface 3 of the support plate to piece of fabric 2,
c) arranging the supporting surface 3 on its supporting position on the object,
d) winding the piece of fabric 2 around the object, and
e) fastening the piece of fabric 2 with itself.

The instruction document 11 may be e.g. in a paper-form or in an electrical form.

The support plate 1 and the piece of fabric 2 included in the kit 200 are preferably shaped and sized for use in a certain indication or body part. The instruction document 11 may include instructions for that specific use. The indication or body part may be selected, for instance, among humerus, wrist, thumb, elbow, knee, ankle, metatarsal fracture, metacarpal fracture, finger, phalanx, forearm, upper arm, neck, and spine. Thus, the use of the kit is simple and does not require skills of highly educated health care personnel.

In one embodiment, the orthopaedic kit 200 is packed and distributed in a package 13. In one embodiment, the package 13 is watertight, thus it is possible to heat the support plate in the package 13 using a water bath without wetting or soaking the support plate.

In one embodiment, the orthopaedic kit 200 comprises at least one accessory 14. The accessory 14 may comprise an additional fastening means, such as a piece of a cohesive tape, for instance.

In one embodiment, the orthopaedic kit 200 comprises an exposing element 9. This element can be arranged for exposing a premature removal of the orthopaedic device 100. In one embodiment, the exposing element 9 also makes it impossible or at least more difficult to remove the orthopaedic device without a correct key, tool, or code. This kind of exposing element 9 may comprise e.g. a plastic belt that is fastened around the orthopaedic device 100 arranged on the object OB. The belt comprises a locking element that cannot be opened without correct key, tool, or code.

In one embodiment, the exposing element 9 comprises an indication marking that breaks or change its appearance when the orthopaedic device 100 is removed. The indication marking may be e.g. a tape or corresponding element, or just a line or other geometrical element drawn/printed on the orthopaedic device 100.

In one embodiment, the exposing element 9 comprises a glue layer or a polymer layer arranged on a suitable surface of the device. Said layer get broken and/or it collects fluff when the device 100 is removed.

The invention is not limited solely to the embodiments described above, but instead many variations are possible within the scope of the inventive concept defined by the claims below. Within the scope of the inventive concept the attributes of different embodiments and applications can be used in conjunction with or replace the attributes of another embodiment or application.

The drawings and the related description are only intended to illustrate the idea of the invention. The invention may vary in detail within the scope of the inventive idea defined in the following claims.

### REFERENCE SYMBOLS

1 support plate
2 piece of fabric
3 supporting surface
4 distal surface
5 self-attaching surface structure
6 receiving zone
7 attachment zone
8 opening
9 exposing element
10 further material layer
11 instruction document
12 overlapping sections
13 package
14 accessory
15 guiding marking
100 orthopaedic device
200 orthopaedic kit
OB object

1. An orthopaedic device, comprising
a support plate,
a piece of fabric, wherein
the support plate is
malleable into an orthopaedically supporting shape, the support plate having a supporting surface to be arranged towards an object to be supported and an opposite distal surface, wherein
the supporting surface of the support plate is adhered to a receiving zone of the piece of fabric, such that
the distal surface of the support plate constitutes a part of the distal surface of the device, and
the piece of fabric is larger than the support plate, providing thus an attachment zone for fastening the orthopaedic device.

2. The orthopaedic device as claimed in claim 1, wherein the support plate is made of a composition comprising
thermoplastic polymer and
wood particles in form of granules, plates and/or fibers.

3. The orthopaedic device as claimed in claim 2, wherein the thermoplastic polymer is non-biodegradable polymer.

4. The orthopaedic device as claimed in claim 2, wherein the thermoplastic polymer is biodegradable polymer.

5. The orthopaedic device as claimed in claim 4, wherein the biodegradable polymer comprises caprolactone homopolymer and the reinforcing material comprises platy wood particles.

6. The orthopaedic device as claimed in claim 1, wherein the support plate is made of a metal or alloy, such as aluminium.

7. The orthopaedic device as claimed in claim 1, wherein the support plate may be solid or comprise at least one opening.

8. The orthopaedic device as claimed in claim 1, wherein
the piece of fabric is made of material comprising thermoplastics, preferably of 100% polyester.

9. The orthopaedic device as claimed in claim 1, wherein the piece of fabric has a self-attaching surface structure having ability to fasten with itself as such.

10. The orthopaedic device as claimed in claim 9, wherein
the self-attaching surface structure of the piece of fabric comprises a fiber loop bundle fabric.

11. The orthopaedic device as claimed in claim 1, wherein the piece of fabric has ability to fasten with itself in a normal temperature, such as in temperature of 20° C.-25° C.

12. The orthopaedic device as claimed in claim 1, comprising an exposing element arranged for exposing a premature removal of the orthopaedic device.

13. The orthopaedic device as claimed in claim 1, comprising a further material layer, such as a layer of fabric, fastened to the receiving zone of the piece of fabric, either on same side with or opposite side thereof in relation to the support plate.

14. An orthopaedic kit, comprising
a support plate, and
a piece of fabric, wherein
the support plate is
malleable into an orthopaedically supporting shape, the support plate having a supporting surface to be arranged towards an object to be supported, and an opposite distal surface, wherein
the size of the piece of fabric is larger than the size of the support plate, wherein the orthopaedic kit further comprises:
an instruction document including instructions for
shaping of the support plate into the orthopaedically supporting shape,
fastening the supporting surface of the support plate to piece of fabric,
arranging the supporting surface on its supporting position on the object, and
winding the piece of fabric around the object.

15. The kit as claimed in claim 14, wherein the support plate and the piece of fabric are shaped and sized for supporting an indication selected from the following indications or body part: humerus, wrist, thumb, elbow, knee, ankle, metatarsal fracture, metacarpal fracture, finger, phalanx, forearm, upper arm, neck, spine.

16. A method for providing temporary orthopaedic support or protection, comprising:
providing a support plate, and a piece of fabric, wherein the support plate is malleable into an orthopaedically supporting shape, the support plate having a supporting surface to be arranged towards an object to be supported, and an opposite distal surface, and wherein the size of the piece of fabric is larger than the size of the support plate,
shaping the support plate into shape of the object to be supported or protected,
fastening the supporting surface of the support plate to the piece of fabric,
arranging the supporting surface on its supporting position on the object so that the piece of fabric is situated between the support plate and the object, and
fastening the support plate on the object by using the piece of fabric.

17. The method as claimed in claim 16, comprising
heating the support plate to a higher temperature, such as to e.g. about 65° C., for at least one of easing shaping thereof and/or attaching to the piece of fabric.
